(19) United States
(12) Patent Application Publication
(10) Pub. No.: US 2016/0136105 A1
(43) Pub. Date: May 19, 2016

(54) Compositions and Methods for the Delivery of Therapeutics

(71) Applicant: Board of Regents of the University of Nebraska, Lincoln, NE (US)

(72) Inventors: Howard E. Gendelman, Omaha, NE (US); Xin-Ming Liu, Columbia, MD (US); Ram S. Veerubhotla, Machilipatnam (IN)

(21) Appl. No.: 14/783,629

(22) PCT Filed: Apr. 11, 2014
(86) PCT No.: PCT/US14/33794

§ 371 Date: Oct. 9, 2015

Related U.S. Application Data
(60) Provisional application No. 61/810,907, filed on Apr. 11, 2013.

(51) Int. Cl. A61K 9/51; A61K 45/06; A61K 31/427

(52) U.S. Cl. CPC A61K 9/5153; A61K 31/427; A61K 45/06

(57) ABSTRACT

The present invention provides compositions and methods for the delivery of antivirals to a cell or subject.

This application is a §371 application of PCT/US2014/033794, filed Apr. 11, 2014, which claims priority under 35 U.S.C. §119(e) to U.S. Provisional Patent Application No. 61/810,907 filed Apr. 11, 2013. The foregoing applications are incorporated by reference herein.

This invention was made with government support under Grant Nos. P01 DA028555, R01 NS034239, P30 MH062261, and P01 MH064570 awarded by National Institutes of Health. The government has certain rights in the invention.

### FIELD OF THE INVENTION

The present invention relates generally to the delivery of therapeutics. More specifically, the present invention relates to compositions and methods for the delivery of therapeutic agents to a patient for the treatment of a viral infection.

### BACKGROUND OF THE INVENTION

The need to improve the bioavailability, pharmacology, cytotoxicities, and interval dosing of antiretroviral medications in the treatment of human immunodeficiency virus (HIV) infection is notable (Broder, S. (2010) Antivir. Res., 85:1-18; Este et al. (2010) Antivir. Res., 85:25-33; Moreno et al. (2010) J. Antimicrob. Chemother., 65:827-835). Since the introduction of antiretroviral therapy (ART), incidences of both mortality and co-morbidities associated with HIV-1 infection have decreased dramatically. It has been demonstrated that nanoformulated indinavir (IDV) can improve biodistribution and antiretroviral efficacy (Dou et al. (2006) Blood 108:2827-2835; Dou et al. (2009) J. Immunol. 183:661-669; Dou et al. (2007) Virology 358:148-158; Nowacek et al. (2009) Nanomedicine 4:903-917). However, many limitations associated with ART still remain which prevent full suppression of viral replication in HIV-infected individuals. These limitations include poor pharmacokinetics (PK) and biodistribution, life-long daily treatment, and multiple untoward toxic side effects (Garvie et al. (2009) J. Adolesc. Health 44:124-132; Hawkins, T. (2006) AIDS Patient Care STDs 20:6-18; Royal et al. (2009) AIDS Care 21:448-455). Since antiretroviral medications are quickly eliminated from the body and do not thoroughly penetrate all organs, dosing schedules tend to be complex and involve large amounts of drug. Patients have difficulty properly following therapy guidelines leading to suboptimal adherence and increased risk of developing viral resistance, which can result in treatment failure and accelerated progression of disease (Danel et al. (2009) J. Infect. Dis. 199:66-76). For HIV-infected patients who also experience psychiatric and mental disorders and/or drug abuse, proper adherence to therapy is even more difficult (Meade et al. (2009) AIDS Patient Care STDs 23:259-266; Baum et al. (2009) J. Acquir. Immune Defic. Syndr., 50:93-99). Accordingly, there is a need for drug delivery systems that optimize cell uptake and retention, improve intracellular stability, extend drug release, maintain antiretroviral efficacy, and minimize cellular toxicity within transporting cells.

### SUMMARY OF THE INVENTION

In accordance with the instant invention, nanoparticles/nanoformulations comprising at least one therapeutic agent and at least one surfactant linked to gp120 are provided. In a particular embodiment, the surfactant is an amphiphilic block copolymer, polysorbate, phospholipid, derivative thereof, or combination thereof. In a particular embodiment, the surfactant is an amphiphilic block copolymer. In a particular embodiment, the nanoparticles/nanoformulations further comprise other surfactants linked to at least one other targeting ligand. An individual nanoparticle may comprise targeted and non-targeted surfactants. In a particular embodiment, the therapeutic agent is an antiviral, antiretroviral, or anti-HIV compound. In a particular embodiment, the surfactant is PLGA-PEG. Pharmaceutical compositions comprising at least nanoparticle of the instant invention and at least one pharmaceutically acceptable carrier are also provided.

According to another aspect of the instant invention, methods for treating, inhibiting, or preventing a disease or disorder (e.g., a retroviral (e.g., HIV) infection) in a subject are provided. In a particular embodiment, the method comprises administering to the subject at least one nanoparticle/nanoformulation of the instant invention. In a particular embodiment, the methods are for treating, inhibiting, or preventing an HIV infection and the therapeutic agent of the nanoparticle is an anti-HIV compound. In a particular embodiment, the method further comprises administering at least one further therapeutic agent or therapy for the disease or disorder, e.g., at least one additional anti-HIV compound.

### BRIEF DESCRIPTIONS OF THE DRAWING

FIG. 1 provides a timecourse of the uptake of gp120 targeted (PLGA-PEG with RTV and gp120) and non-targeted nanoformulations (PLGA-PEG with RTV) into monocyte-derived macrophage (MDM).

FIG. 2 provides a timecourse of the uptake of gp120 targeted (PLGA-PEG with RTV and gp120) in the presence or absence of anti-gp120 antibody and non-targeted nanoformulations (PLGA-PEG with RTV) into monocyte-derived macrophage (MDM).

FIG. 3A provides a graph of the plasma concentration of RTV after administration of 100 mg/kg of the gp120 nanoRTV or non-targeted nanoRTV nanoformulations to mice. Data are means±SEM. FIG. 4B shows the biodistribution of RTV one and seven days after administration of 100 mg/kg of the gp120 nanoRTV or non-targeted nanoRTV nanoformulations to mice. Data are means±SEM.

### DETAILED DESCRIPTION OF THE INVENTION

Antiretroviral therapy (ART) shows several limitations in adherence, pharmacokinetics, effectiveness, and biodistribution while inducing metabolic and cytotoxic aberrations. Administrations commonly require life-long frequent daily dosing, substantive toxicities, and demonstrate limited access to tissue and cellular viral reservoirs. This precludes viral eradication efforts. As there are no current vaccination strategies for HIV eradication, alternative chemical vaccination strategies are desirable. To this end, the instant invention provides HIV sanctuary-targeted long-acting nanoformulated ART to improve patient adherence, reduce systemic toxicities, and reduce residual viral loads. Such long-acting HIV treatments will facilitate lower dosing intervals from daily to monthly or even yearly. The instant invention allows for ART vaccines for the long-term goal of HIV eradication. The invention may also be used as an efficient pre-exposure prophylaxis (PrEP) strategy.

In accordance with the instant invention, HIV gp120 decorated nanoparticles which can be called artificial HIV “virion” nanoparticles are provided which specifically deliver antiretroviral therapies (ART) to virus target cells and tissues. The gp120 nanoparticles resemble the virus itself in size, shape, charge and overall configuration, including the surface protein coat. This allows the nanoparticle to specifically target the exact same sites of viral replication that would be seen by the viral particle itself. Target cells include, without limitation, CD4+ monocytes, macrophages, T lymphocytes, and dendritic cells. The gp120 nanoparticles of the instant invention specifically target sites of both active viral replication and HIV-reservoirs. Accordingly, HIV gp120 nanoparticles specifically deliver ART to HIV target cells and tissue in order to eradicate HIV infection with minimum side effects. Moreover, any secondary immunogenicity towards the HIV gp120 nanoparticles would further stimulate the immune system against HIV and lead to further induction of humoral and cellular immune responses against the virus. The invention can be used for HIV prevention, treatment and/or eradication. The gp120 nanoparticles of the instant invention can prevent ongoing viral replication and prevent any cell to cell spread of virus by bringing appropriate concentrations of antiretroviral drugs to specific target site reservoirs.

The instant invention encompasses nanoparticles/nanoformulations for the delivery of compounds to a cell. In a particular embodiment, the nanoparticle is for the delivery of antiretroviral therapy to a subject. The nanoparticles of the instant invention comprise at least one antiretroviral and at least one surfactant. These components of the nanoparticle, along with other optional components, are described hereinbelow.

Methods of synthesizing the nanoparticles/nanoformulations of the instant invention are known in the art. For example, U.S. Patent Application Publication No. 2013/0236553 provides methods for synthesizing the instant nanoparticles/nanoformulations. In a particular embodiment, the surfactants are firstly chemically modified with targeting ligands and then mixed with non-targeted surfactants in certain molar ratios to coat on the surface of drug suspensions using milling (e.g., wet-milling), homogenization, particle replication in nonwetting template (PRINT) technology, film rehydration, single and/or double emulsions, flash nanoprecipitation, and/or sonication techniques, thereby preparing targeted nanoformulations. In a particular embodiment, the nanoformulations are synthesized using milling and/or homogenization. Targeted nanoformulations using ligands with high molecular weight may be developed through either physically or chemically coating or/and binding on the surface of surfactants or/and drug nanoformulations.

The nanoparticles/nanoformulations of the instant invention may be used to deliver any agent(s) or compound(s), particularly bioactive agents, particularly therapeutic agents or diagnostic agents such as antiviral compounds to a cell or a subject (including non-human animals). The nanoparticles of the instant invention comprise at least one therapeutic agent, particularly at least one antiretroviral. The nanoparticles may be crystalline (solids having the characteristics of crystals) or solid-state nanoparticles of the therapeutic agent, therapeutic agent dispersed in polymer matrix, or therapeutic agent encapsulated polymer/lipid vesicles. In a particular embodiment, the nanoparticles are synthesized by adding the therapeutic agent, particularly the free base form of the therapeutic agent, to a surfactant (described below) solution and then generating the nanoparticles by wet milling or high pressure homogenization. The therapeutic agent and surfactant solution may be agitated prior to wet milling or high pressure homogenization. In a particular embodiment, the nanoparticles are synthesized by single emulsion, extrusion, or film rehydration.

The nanoparticles of the instant invention may be used to deliver any agent(s) or compound(s), particularly bioactive agents (e.g., therapeutic agent or diagnostic agent) to a cell or a subject (including non-human animals). As used herein, the term “bioactive agent” also includes compounds to be screened as potential leads in the development of drugs or plant protecting agents. Bioactive agent and therapeutic agents include, without limitation, polypeptides, peptides, glycoproteins, nucleic acids, synthetic and natural drugs, peptoides, polyenes, macrocyles, glycosides, terpenes, terpenoids, aliphatic and aromatic compounds, small molecules, and their derivatives and salts. In a particular embodiment, the therapeutic agent is a chemical compound such as a synthetic and natural drug. While any type of compound may be delivered to a cell or subject by the compositions and methods of the instant invention, the following description of the inventions exemplifies the compound as a therapeutic agent.

The nanoparticles of the instant invention comprise at least one therapeutic agent. The nanoparticles may be crystalline (solids having the characteristics of crystals) nanoparticles of the therapeutic agent, wherein the nanoparticles comprise about 95% or more (e.g., 99%) pure therapeutic agent. In a particular embodiment, the nanoparticles are synthesized by adding the therapeutic agent, particularly the free base form of the therapeutic agent, to a surfactant (described below) solution and then generating the nanoparticles by wet milling or high pressure homogenization. The therapeutic agent and surfactant solution may be agitated prior the wet milling or high pressure homogenization.

In a particular embodiment, the resultant nanoparticle is up to about 2 or 3 μm in diameter, particularly up to about 1 μm in diameter. In a particular embodiment, the nanoparticle is about 100 nm to about 500 nm in diameter or about 200 to about 350 nm in diameter. The nanoparticles may be, for example, rod shaped, elongated rods, irregular, or round shaped. The nanoparticles of the instant invention may be neutral or charged. The nanoparticles may be charged positively or negatively.

The therapeutic agent may be hydrophobic, a water insoluble compound, or a poorly water soluble compound. For example, the therapeutic agent may have a solubility of less than about 10 mg/ml, less than 1 mg/ml, more particularly less than about 100 μg/ml, and more particularly less than about 25 μg/ml in water or aqueous media in a pH range of 0-14, preferably between pH 4 and 10, particularly at 20° C.

In a particular embodiment, the therapeutic agent of the nanoparticles of the instant invention is an antimicrobial, particularly an antiviral, more particularly an antiretroviral. The antiretroviral may be effective against or specific to lentiviruses. Lentiviruses include, without limitation, human immunodeficiency virus (HIV) (e.g., HIV-1, HIV-2), bovine immunodeficiency virus (BIV), feline immunodeficiency virus (FIV), simian immunodeficiency virus (SIV), and equine infectious anemia virus (EIA). In a particular embodiment, the therapeutic agent is an anti-HIV agent.

An anti-HIV compound or an anti-HIV agent is a compound which inhibits HIV. Examples of an anti-HIV agent include, without limitation:

(I) Nucleoside-analog reverse transcriptase inhibitors (NRTIs). NRTIs refer to nucleosides and nucleotides and analogues thereof that inhibit the activity of HIV-1 reverse transcriptase. Example of a nucleoside-analog reverse transcriptase inhibitors include, without limitation, zidovudine (azidothymidine (AZT)), lamivudine (3TC), abacavir, emtricitabine (FTC), tenofovir, didanosine, stavudine, CMX157, 4′ethynyl-2-fluoro-2′-deoxyadenosine (EFdA), and adefovir dipivoxil.

(II) Non-nucleoside reverse transcriptase inhibitors (NNRTIs). NNRTIs are allosteric inhibitors which bind reversibly at a nonsubstrate-binding site on the HIV reverse transcriptase, thereby altering the shape of the active site or blocking polymerase activity. Examples of NNRTIs include, without limitation, delavirdine (BHAP, U-90152; RESCRIPTOR®), efavirenz (DMP-266, SUSTIVA®), nevirapine (VIRAMUNE®), PNU-142721, capravirine (S-1153, AG-1549), emivirine (+)-calanolide A (NSC-675451) and B, etravirine (TMC-125), rilpivirne (TMC278, Edurant™), doravirine (MK-1439), GSK2248761 (IDX899), DAPY (TMC120), BILR-355 BS, PHI-236, and PHI-443 (TMC-278).

(III) Protease inhibitors (PI). Protease inhibitors are inhibitors of the HIV-1 protease. Examples of protease inhibitors include, without limitation, darunavir, amprenavir (141W94, AGENERASE®), tipranivir (PNU-140690, APTIVUS®), indinavir (MK-639; CRIXIVAN®), saquinavir (INVIRASE®, FORTOVASE®), fosamprenavir (LEXIVA®), lopinavir (ABT-378), ritonavir (ABT-538, NORVIR®), atazanavir (REYATAZ®), nelfinavir (AG-1343, VIRACEPT®), lasinavir (BMS-234475/CGP-61755), BMS-2322623, GW-640385X (VX-385), AG-001859, and SM-309515.

(IV) Fusion or entry inhibitors. Fusion or entry inhibitors are compounds, such as peptides, which act by binding to HIV envelope protein and blocking the structural changes necessary for the virus to fuse with the host cell. Examples of fusion inhibitors include, without limitation, CCR5 receptor antagonists (e.g., maraviroc (Selzentry®, Celsentri)), enfuvirtide (INN, FUZEON®), T-20 (DP-178, FUZEON®) and T-1249.

(V) Integrase inhibitors. Integrase inhibitors are a class of antiretroviral drug designed to block the action of integrase, a viral enzyme that inserts the viral genome into the DNA of the host cell. Examples of integrase inhibitors include, without limitation, raltegravir, elvitegravir, dolutegravir, GSK1265744, and MK-2048.

Anti-HIV compounds also include maturation inhibitors. Anti-HIV compounds also include HIV vaccines such as, without limitation, ALVAC® HIV (vCP1521), AIDSVAX®B/E (gp120), and combinations thereof. Anti-HIV compounds also include HIV antibodies (e.g., antibodies against gp160, gp120 and/or gp41), particularly broadly neutralizing antibodies.

More than one anti-HIV agent may be used, particularly where the agents have different mechanisms of action (as outlined above). In a particular embodiment, the anti-HIV therapy is highly active antiretroviral therapy (HAART).

In a particular embodiment, the anti-HIV agent is hydrophobic. In a particular embodiment, the anti-HIV agent of the instant invention is a protease inhibitor, NNRTI, or NRTI, particularly a protease inhibitor (e.g., indinavir, ritonavir, atazanavir, or efarirenz).

As stated hereinabove, the nanoparticles of the instant invention comprise at least one surfactant. A “surfactant” refers to a surface-active agent, including substances commonly referred to as wetting agents, detergents, dispersing agents, or emulsifying agents. Surfactants are usually organic compounds that are amphiphilic.

Examples of surfactants include, without limitation, synthetic or natural phospholipids, pegylated lipids, polysorbates, poly(ethylene glycol)-co-poly(lactide-co-glycolide) (PEG-PLGA), their derivatives, ligand-conjugated derivatives and combinations thereof. Other surfactants and their combinations that can form stable nanosuspensions or/and can chemically/physically bind to the targeting ligands of HIV infectable/infected CD4+ T cells, macrophages and dendritic cells can be used in the instant invention. Further examples of surfactants include, without limitation (inclusive of combinations of hydrophobic and hydrophilic blocks of the following): 1) nonionic surfactants (e.g., functionalized polyesters such as pegylated and/or polysaccharide-conjugated polyesters and other hydrophobic polymeric blocks such as poly(lactide-co-glycolide) (PLGA), polylactic acid (PLA), poly(glycolic acid), polycaprolactone (PCL), other polyesters, poly(propylene oxide), poly(1,2-butylene oxide), poly(n-butylene oxide), poly(tetrahydrofurane), and poly(styrene); glyceryl esters, polyoxyethylene fatty alcohol ethers, polyoxyethylene sorbitan fatty acid esters, polyoxyethylene fatty acid esters, sorbitan esters, glycerol monostearate, polyethylene glycols, polypropyleneglycols, cetyl alcohol, cetostearyl alcohol, stearyl alcohol, aryl alkyl polyether alcohols, polyoxyethylene-polyoxypropylene copolymers, poloxamines, cellulose, methylcellulose, hydroxylmethylcellulose, hydroxypropylcellulose, hydroxypropylmethylcellulose, polysaccharides, starch and their derivatives, hydroxyethylstarch, polyvinyl alcohol, polyvinylpyrrolidone, and their combination thereof); and 2) ionic surfactants (e.g., phospholipids, amphiphilic lipids, 1,2-dialkylglycero-3-alkylphophocholines, dimethylaminoethanecarbamoyl cheolesterol (DC-Chol), N-[1-(2,3-Dioleoyloxy)propyl]-N,N,N-trimethylammonium (DOTAP), alkyl pyridinium halides, quaternary ammonium compounds, lauryldimethylbenzylammonium, acyl carnitine hydrochlorides, dimethyldioctadecylammonium (DDAB), n-octylamines, oleylamines, benzalkonium, cetyltrimethylammonium, chitosan, chitosan salts, poly(ethylenimine) (PEI), poly(N-isopropyl acrylamide (PNIPAM), and poly(allylamine) (PAH), poly(dimethyldiallylammonium chloride) (PDDA), alkyl sulfonates, alkyl phosphates, alkyl phosphonates, potassium laurate, triethanolamine stearate, sodium lauryl sulfate, sodium dodecylsulfate, alkyl polyoxyethylene sulfates, alginic acid, alginic acid salts, hyaluronic acid, hyaluronic acid salts, gelatins, dioctyl sodium sulfosuccinate, sodium carboxymethylcellulose, cellulose sulfate, dextran sulfate and carboxymethylcellulose, chondroitin sulfate, heparin, synthetic poly(acrylic acid) (PAA), poly(methacrylic acid) (PMA), poly(vinyl sulfate) (PVS), poly(styrene sulfonate) (PSS), bile acids and their salts, cholic acid, deoxycholic acid, glycocholic acid, taurocholic acid, glycodeoxycholic acid, and combinations thereof).

In a particular embodiment of the invention, the surfactant is present in the nanoparticle and/or surfactant solution to synthesize the nanoparticle (as described herein) at a concentration ranging from about 0.0001% to about 5%. In a particular embodiment, the concentration of the surfactant ranges from about 0.1% to about 2%. In a particular embodiment, the nanoparticle comprises about 1% to about 99% or higher of therapeutic agent, particularly about 5% to about 99% or about 5% to about 95%. In a particular embodiment, the nanoparticle comprises a high amount of therapeutic agent, particularly at least about 50%, 75%, 80%, 85%, 90%, 95%, 97%, 98%, 99% or higher of therapeutic agent by weight.

The surfactant of the instant invention may be charged or neutral. In a particular embodiment, the surfactant is neutral or negatively charged (e.g., poloxamers, polysorbates, phospholipids, and their derivatives).

In a particular embodiment, the surfactant is an amphiphilic block copolymer. In a particular embodiment, the hydrophilic block of the amphiphilic block copolymer is poly(ethylene oxide) or polysaccharide. In a particular embodiment, the hydrophobic block of the amphiphilic block copolymer is selected from the group consisting of polyester, polyanhydride, poly(propylene oxide), poly(1,2-butylene oxide), poly(n-butylene oxide), poly(tetrahydrofurane), poly(styrene), functionalized polyesters, poly(lactic acid), poly(glycolic acid), poly(lactic-coglycolic acid), polycaprolactone, and functionalized poloxamers.

In a particular, embodiment, at least one surfactant of the nanoparticle is an amphiphilic block copolymer, particularly a copolymer comprising at least one block of poly(oxyethylene) and at least one block of poly(oxypropylene). In a particular embodiment, the surfactant is poloxamer 407. Amphiphilic block copolymers are exemplified by the block copolymers having the formulas:

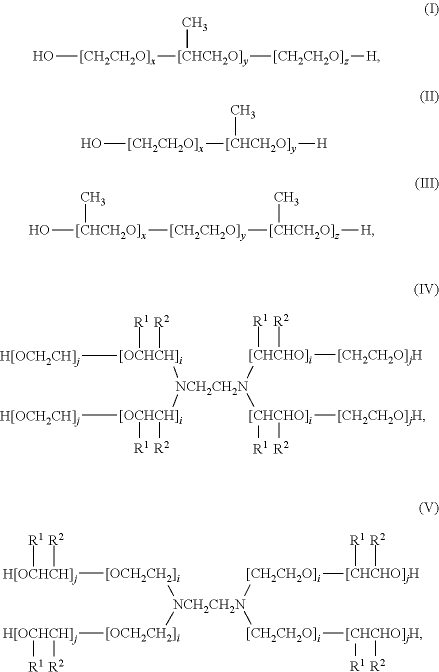

in which x, y, z, i, and j have values from about 2 to about 800, preferably from about 5 to about 200, more preferably from about 5 to about 80, and wherein for each R¹, R² pair, as shown in formula (IV) and (V), one is hydrogen and the other is a methyl group. The ordinarily skilled artisan will recognize that the values of x, y, and z will usually represent a statistical average and that the values of x and z are often, though not necessarily, the same. Formulas (I) through (III) are oversimplified in that, in practice, the orientation of the isopropylene radicals within the B block will be random. This random orientation is indicated in formulas (IV) and (V), which are more complete. Such poly(oxyethylene)-poly(oxypropylene) compounds have been described by Santon (Am. Perfumer Cosmet. (1958) 72(4):54-58); Schmolka (Loc. cit. (1967) 82(7):25-30), Schick, ed. (Non-ionic Suifactants, Dekker, N.Y., 1967 pp. 300-371). A number of such compounds are commercially available under such generic trade names as “lipoloxamers”, “Pluronics®,” “poloxamers,” and “synperonics.” Pluronic® copolymers within the B-A-B formula, as opposed to the A-B-A formula typical of Pluronics®, are often referred to as “reversed” Pluronics®, “Pluronic® R” or “meroxapol.” Generally, block copolymers can be described in terms of having hydrophilic “A” and hydrophobic “B” block segments. Thus, for example, a copolymer of the formula A-B-A is a triblock copolymer consisting of a hydrophilic block connected to a hydrophobic block connected to another hydrophilic block. The “polyoxamine” polymer of formula (IV) is available from BASF under the tradename Tetronic®. The order of the polyoxyethylene and polyoxypropylene blocks represented in formula (IV) can be reversed, creating Tetronic R®, also available from BASF (see, Schmolka, J. Am. Oil. Soc. (1979) 59:110).

Polyoxypropylene-polyoxyethylene block copolymers can also be designed with hydrophilic blocks comprising a random mix of ethylene oxide and propylene oxide repeating units. To maintain the hydrophilic character of the block, ethylene oxide can predominate. Similarly, the hydrophobic block can be a mixture of ethylene oxide and propylene oxide repeating units. Such block copolymers are available from BASF under the tradename Pluradot™. Poly(oxyethylene)-poly(oxypropylene) block units making up the first segment need not consist solely of ethylene oxide. Nor is it necessary that all of the B-type segment consist solely of propylene oxide units. Instead, in the simplest cases, for example, at least one of the monomers in segment A may be substituted with a side chain group.

A number of poloxamer copolymers are designed to meet the following formula:

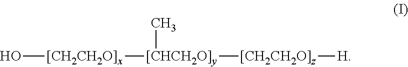

Examples of poloxamers include, without limitation, Pluronic® L31, L35, F38, L42, L43, L44, L61, L62, L63, L64, P65, F68, L72, P75, F77, L81, P84, P85, F87, F88, L92, F98, L101, P103, P104, P105, F108, L121, L122, L123, F127, 10R5, 10R8, 12R3, 17R1, 17R2, 17R4, 17R8, 22R4, 25R1, 25R2, 25R4, 25R5, 25R8, 31R1, 31R2, and 31R4. Pluronic® block copolymers are designated by a letter prefix followed by a two or a three digit number. The letter prefixes (L, P, or F) refer to the physical form of each polymer, (liquid, paste, or flakeable solid). The numeric code defines the structural parameters of the block copolymer. The last digit of this code approximates the weight content of EO block in tens of weight percent (for example, 80% weight if the digit is 8, or 10% weight if the digit is 1). The remaining first one or two digits encode the molecular mass of the central PO block. To decipher the code, one should multiply the corresponding number by 300 to obtain the approximate molecular mass in daltons (Da). Therefore Pluronic nomenclature provides a convenient approach to estimate the characteristics of the block copolymer in the absence of reference literature. For example, the code ‘F127’ defines the block copolymer, which is a solid, has a PO block of 3600 Da (12×300) and 70% weight of EO. The precise molecular characteristics of each Pluronic® block copolymer can be obtained from the manufacturer.

Other biocompatible amphiphilic copolymers include those described in Gaucher et al. (J. Control Rel. (2005) 109:169-188. Examples of other polymers include, without limitation, poly(2-oxazoline) amphiphilic block copolymers, polyethylene glycol-polylactic acid (PEG-PLA), PEG-PLA-PEG, polyethylene glycol-poly(lactide-co-glycolide) (PEG-PLG), polyethylene glycol-poly(lactic-co-glycolic acid) (PEG-PLGA), polyethylene glycol-polycaprolactone (PEG-PCL), polyethylene glycol-polyaspartate (PEG-PAsp), polyethylene glycol-poly(glutamic acid) (PEG-PG1u), polyethylene glycol-poly(acrylic acid) (PEG-PAA), polyethylene glycol-poly(methacrylic acid) (PEG-PMA), polyethylene glycol-poly(ethyleneimine) (PEG-PEI), polyethylene glycol-poly(L-lysine) (PEG-PLys), polyethylene glycol-poly(2-(N,N-dimethylamino)ethyl methacrylate) (PEG-PDMAEMA) and polyethylene glycol-Chitosan derivatives. In a particular embodiment, the amphiphilic copolymer is PEG-PLGA.

The nanoparticles/nanoformulations of the instant invention may comprise targeted and non-targeted surfactants. In a particular embodiment, the molar ratio of targeted and non-targeted surfactants in the nanoparticles/nanoformulations of the instant invention is from about 0.001 to 100%. Typically, the nanoparticles/nanoformulations of the instant invention will comprise more non-targeted surfactants than targeted surfactants (e.g., a ratio of at least about 1:3, at least about 1:5, at least about 1:10 or more for targeted surfactant:non-targeted surfactant). In a particular embodiment, the nanoparticles/nanoformulations of the instant invention comprise an envelope (env) protein (e.g., HIV gp160), particularly an env surface protein (e.g., HIV gp120 such as HIV-1 gp120) conjugated surfactant and a non-targeted version of the surfactant. The HIV env gene encodes the viral envelope glycoprotein that is translated as a 160 kDa precursor (gp160) which is cleaved into a 120 kDa surface/external envelope glycoprotein (gp120) and a 41 kDa transmembrane envelope glycoprotein (gp41). The gp120 of the instant invention may be modified (e.g., glycosylated). The gp120 may be linked directly to the surfactant or via a linker. Examples of chemical strategies for conjugating gp120 to the surfactant include, without limitation, maleimide conjugation, amine conjugation, carboxy conjugation, cysteine modification, oxidized carbohydrates or N-terminus, and click chemistry.

The gp120 can be from any HIV isolate (e.g., any primary or cultured HIV-1 or HIV-2 isolate, strain, or clade). HIV isolates are classified into discrete genetic subtypes. For example, examples of HIV-1 subtypes include, without limitation: A1, A2, A3, A4, B, C, D, E, F1, F2, G, H, J and K (see, e.g., Taylor et al. (2008) NEJM, 359:1965-1966). GenBank Gene ID: 155971 and GenBank Accession Nos. NP_057856 and NP_579894 provide example sequences of env and gp120. While the gp120 exemplified herein is from HIV, the envelope surface protein may be from any retrovirus, particularly any lentivirus such as bovine immunodeficiency virus (BIV), feline immunodeficiency virus (FIV), simian immunodeficiency virus (SIV), or equine infectious anemia virus (EIA).

Generally, the linker is a chemical moiety comprising a covalent bond or a chain of atoms that covalently attaches the ligand to the surfactant. The linker can be linked to any synthetically feasible position of gp120 and the surfactant. Exemplary linkers may comprise at least one optionally substituted; saturated or unsaturated; linear, branched or cyclic alkyl group or an optionally substituted aryl group. The linker may also be a polypeptide (e.g., from about 1 to about 10 amino acids, particularly about 1 to about 5). The linker may be non-degradable and may be a covalent bond or any other chemical structure which cannot be substantially cleaved or cleaved at all under physiological environments or conditions. In a particular embodiment, the linker is maleimide (or residue thereof). In a particular embodiment, the nanoparticles/nanoformulations of the instant invention comprise PLGA-PEG-gp120 and PLGA-PEG.

The nanoparticles/nanoformulations of the instant invention may further comprise one or more other targeted surfactants in addition to the gp120 conjugated surfactant. The surfactant of the instant invention may be linked to a targeting ligand. A targeting ligand is a compound that will specifically bind to a specific type of tissue or cell type. In a particular embodiment, the targeting ligand is a ligand for a cell surface marker/receptor. The targeting ligand may be an antibody or fragment thereof immunologically specific for a cell surface marker (e.g., protein or carbohydrate) preferentially or exclusively expressed on the targeted tissue or cell type. The targeting ligand may be linked directly to the surfactant or via a linker. In a particular embodiment, the targeting ligand directs the nanoparticles to HIV tissue and cellular sanctuaries/reservoirs (e.g., central nervous system, gut associated lymphoid tissues (GALT), CD4+ T cells, macrophages, dendritic cells, etc.). In a particular embodiment, the targeting ligand is viral (e.g., HIV) envelope protein or other viral protein that mediates the entry of the virus (e.g., HIV) into cells In a particular embodiment, the targeting ligand is a macrophage targeting ligand; CD4+ T cell targeting ligand, or a dendritic cell targeting ligand. Macrophage and/or monocyte targeting ligands include, without limitation, folate receptor ligands (e.g., folate (folic acid) and folate receptor ligands or antibodies and fragments thereof (see, e.g., Sudimack et al. (2000) Adv. Drug Del. Rev., 41:147-162)), mannose receptor ligands (e.g., mannose), formyl peptide receptor (FPR) ligands (e.g., N-formyl-Met-Leu-Phe (fMLF)), and tuftsin (the tetrapeptide Thr-Lys-Pro-Arg). Other targeting ligands (e.g., for targeting HIV reservoirs) include, without limitation, hyaluronic acid, and ligands or antibodies specific for CD4, CCR5, CXCR4, CD7, CD 111, CD204, CD49a, or CD29. As demonstrated herein, the targeting of the nanoparticles (e.g., to macrophage) provides for superior targeting, decreased excretion rates, decreased toxicity, and prolonged half life compared to free drug or non-targeted nanoparticles.

The instant invention encompasses pharmaceutical compositions comprising at least one nanoparticle of the instant invention (sometimes referred to herein as nanoART) and at least one pharmaceutically acceptable carrier. As stated hereinabove, the nanoparticle may comprise more than one therapeutic agent. In a particular embodiment, the pharmaceutical composition comprises a first nanoparticle comprising a first therapeutic agent(s) and a second nanoparticle comprising a second therapeutic agent(s), wherein the first and second therapeutic agents are different. The pharmaceutical compositions of the instant invention may further comprise other therapeutic agents (e.g., other anti-HIV compounds (e.g., those described hereinabove)).

The present invention also encompasses methods for preventing, inhibiting, and/or treating a microbial infection, particularly a viral infection, particularly retroviral or lentiviral infections, particularly HIV infections (e.g., HIV-1). The pharmaceutical compositions of the instant invention can be administered to an animal, in particular a mammal, more particularly a human, in order to treat/inhibit an HIV infection. The pharmaceutical compositions of the instant invention may also comprise at least one other antiviral agent, particularly at least one other anti-HIV compound/agent. The additional anti-HIV compound may also be administered in a separate pharmaceutical composition from the anti-HIV nanoparticles of the instant invention. The pharmaceutical compositions may be administered at the same time or at different times (e.g., sequentially).

The dosage ranges for the administration of the pharmaceutical compositions of the invention are those large enough to produce the desired effect (e.g., curing, relieving, treating, and/or preventing the HIV infection, the symptoms of it (e.g., AIDS, ARC), or the predisposition towards it). In a particular embodiment, the pharmaceutical composition of the instant invention is administered to the subject at an amount from about 5 μg/kg to about 500 mg/kg. In a particular embodiment, the pharmaceutical composition of the instant invention is administered to the subject at an amount greater than about 5 μg/kg, greater than about 50 μg/kg, greater than about 0.1 mg/kg, greater than about 0.5 mg/kg, greater than about 1 mg/kg, or greater than about 5 mg/kg. In a particular embodiment, the pharmaceutical composition of the instant invention is administered to the subject at an amount from about 0.5 mg/kg to about 100 mg/kg, about 10 mg/kg to about 100 mg/kg, or about 15 mg/kg to about 50 mg/kg. The dosage should not be so large as to cause significant adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counter indications.

The nanoparticles described herein will generally be administered to a patient as a pharmaceutical composition. The term “patient” as used herein refers to human or animal subjects. These nanoparticles may be employed therapeutically, under the guidance of a physician.

The pharmaceutical compositions comprising the nanoparticles of the instant invention may be conveniently formulated for administration with any pharmaceutically acceptable carrier(s). For example, the complexes may be formulated with an acceptable medium such as water, buffered saline, ethanol, polyol (for example, glycerol, propylene glycol, liquid polyethylene glycol and the like), dimethyl sulfoxide (DMSO), oils, detergents, suspending agents, or suitable mixtures thereof. The concentration of the nanoparticles in the chosen medium may be varied and the medium may be chosen based on the desired route of administration of the pharmaceutical composition. Except insofar as any conventional media or agent is incompatible with the nanoparticles to be administered, its use in the pharmaceutical composition is contemplated.

The dose and dosage regimen of nanoparticles according to the invention that are suitable for administration to a particular patient may be determined by a physician considering the patient's age, sex, weight, general medical condition, and the specific condition for which the nanoparticles are being administered and the severity thereof. The physician may also take into account the route of administration, the pharmaceutical carrier, and the nanoparticle's biological activity.

Selection of a suitable pharmaceutical composition will also depend upon the mode of administration chosen. For example, the nanoparticles of the invention may be administered by direct injection or intravenously. In this instance, a pharmaceutical composition comprises the nanoparticle dispersed in a medium that is compatible with the site of injection.

Nanoparticles of the instant invention may be administered by any method. For example, the nanoparticles of the instant invention can be administered, without limitation parenterally, subcutaneously, orally, topically, pulmonarily, rectally, vaginally, intravenously, intraperitoneally, intrathecally, intracerbrally, epidurally, intramuscularly, intradermally, or intracarotidly. In a particular embodiment, the nanoparticles are administered intraperitoneally, intravenously, intramuscularly or subcutaneously. Pharmaceutical compositions for injection are known in the art. If injection is selected as a method for administering the nanoparticle, steps must be taken to ensure that sufficient amounts of the molecules or cells reach their target cells to exert a biological effect. Dosage forms for oral administration include, without limitation, tablets (e.g., coated and uncoated, chewable), gelatin capsules (e.g., soft or hard), lozenges, troches, solutions, emulsions, suspensions, syrups, elixirs, powders/granules (e.g., reconstitutable or dispersible) gums, and effervescent tablets. Dosage forms for parenteral administration include, without limitation, solutions, emulsions, suspensions, dispersions and powders/granules for reconstitution. Dosage forms for topical administration include, without limitation, creams, gels, ointments, salves, patches and transdermal delivery systems.

Pharmaceutical compositions containing a nanoparticle of the present invention as the active ingredient in intimate admixture with a pharmaceutically acceptable carrier can be prepared according to conventional pharmaceutical compounding techniques. The carrier may take a wide variety of forms depending on the form of pharmaceutical composition desired for administration, e.g., intravenous, oral, direct injection, intracranial, and intravitreal.

A pharmaceutical composition of the invention may be formulated in dosage unit form for ease of administration and uniformity of dosage. Dosage unit form, as used herein, refers to a physically discrete unit of the pharmaceutical composition appropriate for the patient undergoing treatment. Each dosage should contain a quantity of active ingredient calculated to produce the desired effect in association with the selected pharmaceutical carrier. Procedures for determining the appropriate dosage unit are well known to those skilled in the art. In a particular embodiment, the nanoformulations of the instant invention may be administered, for example, daily or once every 2, 3, 4, 5, 6, or 7 days or may be administered weekly or once every 2, 3, or 4 weeks. In a particular embodiment, the nanoformulations of the instant invention, due to their long-acting therapeutic effect, may be administered once every 6 or 12 months or even less frequently. For example, the nanoformulations of the instant invention may be administered once every 1, 2, 3, 4, 5, 6, 7, 8, 9, 10, 11, 12, 15, 18, 21, 24, or more months.

Dosage units may be proportionately increased or decreased based on the weight of the patient. Appropriate concentrations for alleviation of a particular pathological condition may be determined by dosage concentration curve calculations, as known in the art.

In accordance with the present invention, the appropriate dosage unit for the administration of nanoparticles may be determined by evaluating the toxicity of the molecules or cells in animal models. Various concentrations of nanoparticles in pharmaceutical composition may be administered to mice, and the minimal and maximal dosages may be determined based on the beneficial results and side effects observed as a result of the treatment. Appropriate dosage unit may also be determined by assessing the efficacy of the nanoparticle treatment in combination with other standard drugs. The dosage units of nanoparticle may be determined individually or in combination with each treatment according to the effect detected.

The pharmaceutical composition comprising the nanoparticles may be administered at appropriate intervals until the pathological symptoms are reduced or alleviated, after which the dosage may be reduced to a maintenance level. The appropriate interval in a particular case would normally depend on the condition of the patient.

The instant invention encompasses methods of treating a disease/disorder comprising administering to a subject in need thereof a pharmaceutical composition comprising a nanoparticle of the instant invention and, preferably, at least one pharmaceutically acceptable carrier. The instant invention also encompasses methods wherein the subject is treated via ex vivo therapy. In particular, the method comprises removing cells from the subject, exposing/contacting the cells in vitro to the nanoparticles of the instant invention, and returning the cells to the subject. In a particular embodiment, the cells comprise macrophage. Other methods of treating the disease or disorder may be combined with the methods of the instant invention may be co-administered with the pharmaceutical compositions of the instant invention.

The instant also encompasses delivering the nanoparticle of the instant invention to a cell in vitro (e.g., in culture). The nanoparticle may be delivered to the cell in at least one carrier.

### Definitions

The following definitions are provided to facilitate an understanding of the present invention.

The singular forms “a,” “an,” and “the” include plural referents unless the context clearly dictates otherwise. “Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., Tween 80, Polysorbate 80), emulsifier, buffer (e.g., Tris HCl, acetate, phosphate), antimicrobial, bulking substance (e.g., lactose, mannitol), excipient, auxiliary agent or vehicle with which an active agent of the present invention is administered. Pharmaceutically acceptable carriers can be sterile liquids, such as water and oils, including those of petroleum, animal, vegetable or synthetic origin. Water or aqueous saline solutions and aqueous dextrose and glycerol solutions are preferably employed as carriers, particularly for injectable solutions. Suitable pharmaceutical carriers are described in “Remington's Pharmaceutical Sciences” by E. W. Martin (Mack Publishing Co., Easton, Pa.); Gennaro, A. R., Remington: The Science and Practice of Pharmacy, (Lippincott, Williams and Wilkins); Liberman, et al., Eds., Pharmaceutical Dosage Forms, Marcel Decker, New York, N.Y.; and Kibbe, et al., Eds., Handbook of Pharmaceutical Excipients, American Pharmaceutical Association, Washington.

The term “treat” as used herein refers to any type of treatment that imparts a benefit to a patient afflicted with a disease, including improvement in the condition of the patient (e.g., in one or more symptoms), delay in the progression of the condition, etc. In a particular embodiment, the treatment of a retroviral infection results in at least an inhibition/reduction in the number of infected cells.

A “therapeutically effective amount” of a compound or a pharmaceutical composition refers to an amount effective to prevent, inhibit, treat, or lessen the symptoms of a particular disorder or disease. The treatment of a microbial infection (e.g., HIV infection) herein may refer to curing, relieving, and/or preventing the microbial infection, the symptom(s) of it, or the predisposition towards it.

As used herein, the term “therapeutic agent” refers to a chemical compound or biological molecule including, without limitation, nucleic acids, peptides, proteins, and antibodies that can be used to treat a condition, disease, or disorder or reduce the symptoms of the condition, disease, or disorder.

As used herein, the term “small molecule” refers to a substance or compound that has a relatively low molecular weight (e.g., less than 4,000, less than 2,000, particularly less than 1 kDa or 800 Da). Typically, small molecules are organic, but are not proteins, polypeptides, or nucleic acids, though they may be amino acids or dipeptides.

The term “antimicrobials” as used herein indicates a substance that kills or inhibits the growth of microorganisms such as bacteria, fungi, viruses, or protozoans.

As used herein, the term “antiviral” refers to a substance that destroys a virus or suppresses replication (reproduction) of the virus.

As used herein, the term “highly active antiretroviral therapy” (HAART) refers to HIV therapy with various combinations of therapeutics such as nucleoside reverse transcriptase inhibitors, non-nucleoside reverse transcriptase inhibitors, HIV protease inhibitors, and fusion inhibitors.

As used herein, the term “amphiphilic” means the ability to dissolve in both water and lipids/apolar environments. Typically, an amphiphilic compound comprises a hydrophilic portion and a hydrophobic portion. “Hydrophobic” designates a preference for apolar environments (e.g., a hydrophobic substance or moiety is more readily dissolved in or wetted by non-polar solvents, such as hydrocarbons, than by water). As used herein, the term “hydrophilic” means the ability to dissolve in water.

As used herein, the term “polymer” denotes molecules formed from the chemical union of two or more repeating units or monomers. The term “block copolymer” most simply refers to conjugates of at least two different polymer segments, wherein each polymer segment comprises two or more adjacent units of the same kind.

An “antibody” or “antibody molecule” is any immunoglobulin, including antibodies and fragments thereof (e.g., scFv), that binds to a specific antigen. As used herein, antibody or antibody molecule contemplates intact immunoglobulin molecules, immunologically active portions of an immunoglobulin molecule, and fusions of immunologically active portions of an immunoglobulin molecule.

As used herein, the term “immunologically specific” refers to proteins/polypeptides, particularly antibodies, that bind to one or more epitopes of a protein or compound of interest, but which do not substantially recognize and bind other molecules in a sample containing a mixed population of antigenic biological molecules.

The following example provides illustrative methods of practicing the instant invention, and is not intended to limit the scope of the invention in any way.

### EXAMPLE

Gp120-nanoART nanoparticles were synthesized by conjugating a poly(lactic-co-glycolic)-b-poly(ethylene glycol)polymer (PLGA-PEG) to gp 120. More specifically, a maleimide functionalized PLGA-PEG (PLGA-PEG-Mal; 20 kDa-5kDa) was utilized. The maleimide group allows for conjugation to the thiol group of cysteines within gp120, thereby forming C—S bonds.

Briefly, ritonavir (RTV) loaded nanoparticles were prepared by the single emulsion technique. The nanoparticles were formed with maleimide-PEG-PLGA (5K-20K). In particular, a 1:10 ratio of maleimide-PEG-PLGA (5K-20K) and PEG-PLGA (5k-20K) was used for formulation preparation. Firstly, a weighed amount of PEO-PLGA, maleimide-PEO-PLGA and RTV A were dissolved in dichloromethane (oil phase) with a weight ratio of polymer to RTV of 1:5. Second, the aqueous phase was 0.5% polyvinyl alcohol (PVA). The oil phase was added to the aqueous phase dropwise, with constant stirring and then sonicated for 60 seconds followed by a 20 second break on an ice bath. This procedure was repeated for three cycles. Dichloromethane was then removed by stirring overnight. Third, the particle suspension was centrifuged at 200×g for 5 minutes. The supernatant fluids were collected to remove the aggregated nanoparticles. A high-speed 50,000×g centrifugation for 30 minutes was used to collect the nanoparticles. After washing twice with phosphate-buffered saline (PBS), the nanoparticles were resuspended into PBS containing gp120 (isolated from HIV infected macrophages or T cells or genetically produced from bacteria). The conjugation was performed at cold room overnight. Unreacted maleimide—in both gp120 and non-targeted formulations—was quenched with cysteine, and non-conjugated gp120 was removed by centrifugation (50,000×g for 30 minutes). The pellets were collected and characterized by dynamic light scattering (Malvern Zetasizer Nano Series Nano-ZS, Malvern Instruments, MA, USA) and then diluted in ultrapure water related to mass concentrations and dispersions. Table 1 shows the physiochemical characterization of various constructs, wherein coumarin 6 is a fluorescent dye.

TABLE 1
Physicochemical characterization of targeted (PLGA-PEG-
Mal gp 120) and non-targeted PLGA nanoformulations.

| | | Zeta | Polydis- |
|---|---|---|---|
| | Size | Potential | persity |
| Name | (nm) | (mV) | Index (PDI) |
| PLGA-PEG-Mal-RTV | 164.8 | −18.3 | 0.106 |
| PLGA-PEG-Mal-RTV-coumarin 6 | 184.9 | −26.3 | 0.096 |
| PLGA-PEG-Mal:PLGA-PEG (1:10) | 202.0 | −28.2 | 0.175 |
| PLGA-PEG-Mal (1:10) gp 120 | 242.4 | −35.8 | 0.217 |

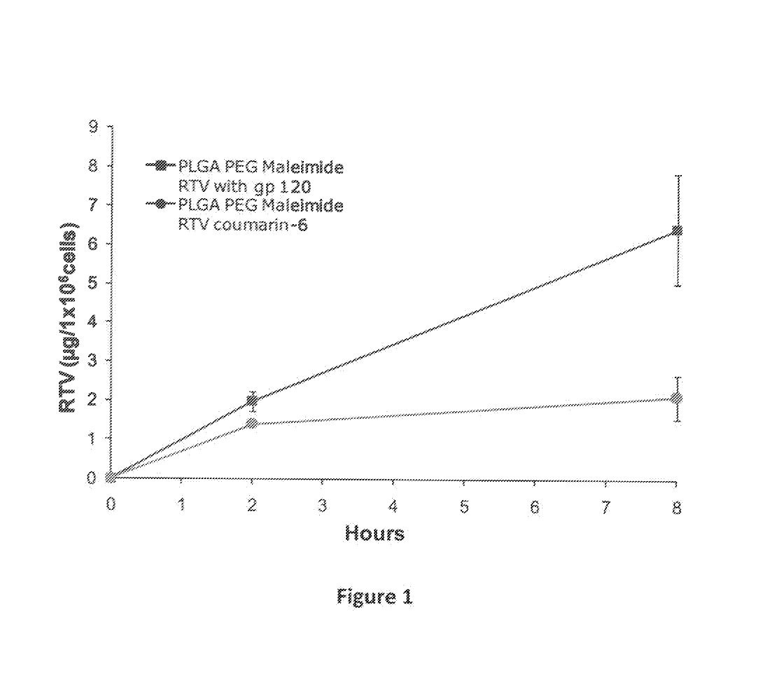

FIG. 1 shows the uptake of gp120 nanoRTV and non-targeted nanoRTV by monocyte derived macrophages (MDM). Specifically, after 7 days of differentiation, monocyte-derived macrophages (MDM) (n=3) were treated with 100 μM RTV for 8 hours (2 hour and 8 hour time points were taken) without media change. Adherent MDM were washed with phosphate buffered saline (PBS) and collected by scraping into PBS. Cells were pelleted by centrifugation at 950×g for 8 minutes at 4° C. Cell pellets were briefly sonicated in methanol and centrifuged at 4° C. The methanol extract was stored at −80° C. until HPLC analysis to determine the amount of RTV.

RTV retention in MDM was also measured and gp120 targeted nanoRTV led to greater retention of RTV in MDM compared to non-targeted nanoRTV (PLGA-PEG-Mal-coumarin 6 with RTV or PLGA-PEG with RTV).

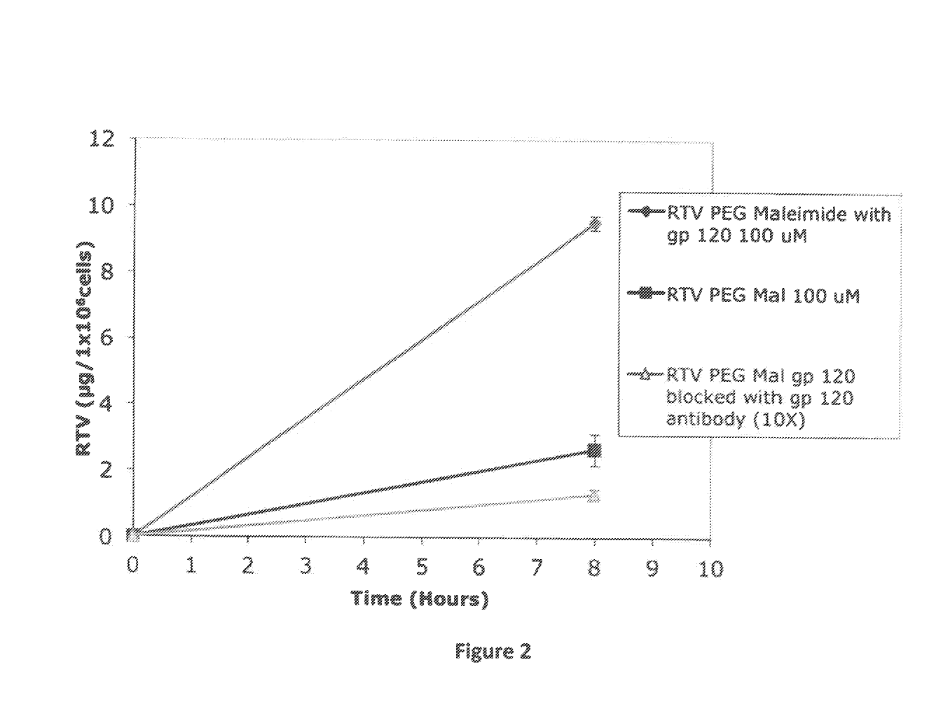

FIG. 2 shows that the uptake of gp120 nanoRTV is blocked by anti-gp120 antibodies. These results indicate that the gp120 of the nanoformulations is mediating entry of the particles into cells.

The pharmacokinetics and biodistribution (PK/BD) of gp120 nanoRTV and non-targeted nanoRTV was studied in mice. Briefly, immunodeficient NOD/SCID-IL2rγ^{null} (NSG) mice, which completely lack functional T-cells, B-cells, NK cells, macrophages, and DC, were engrafted with human cells to reconstitute the immune system including peripheral blood lymphocytes (PBL) (Ishikawa et al. (2005) Blood 106:1565-73; Shultz et al. (2000) J. Immunol., 164:2496-2507). 100 mg/kg RTV as targeted or non-targeted nanoRTV was administered intraperitoneally at Day 0 and plasma and tissue drug levels were determined at various times thereafter. Drug levels determined by ultra performance liquid chromatography—tandem mass spectrometer (UPLC-MS/MS).

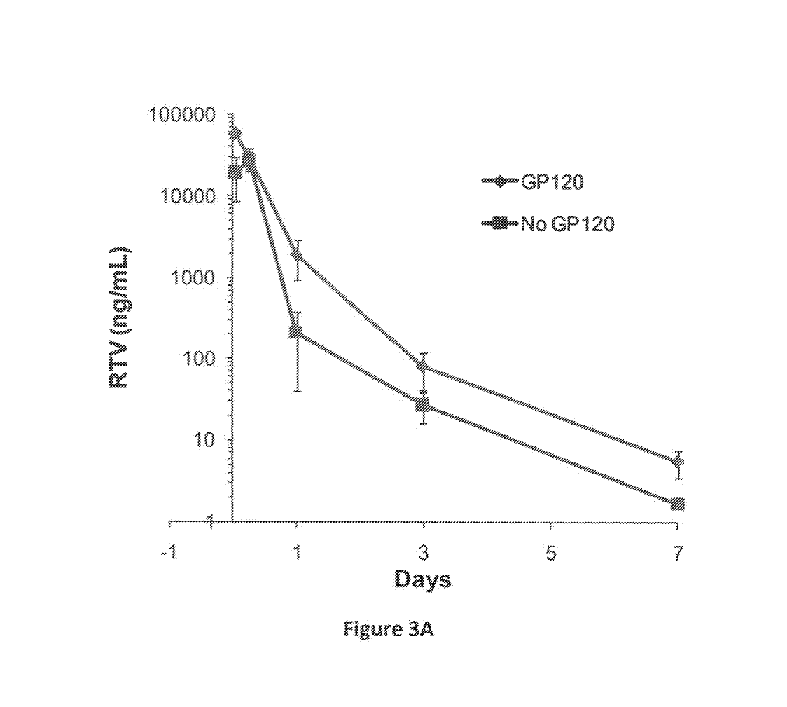

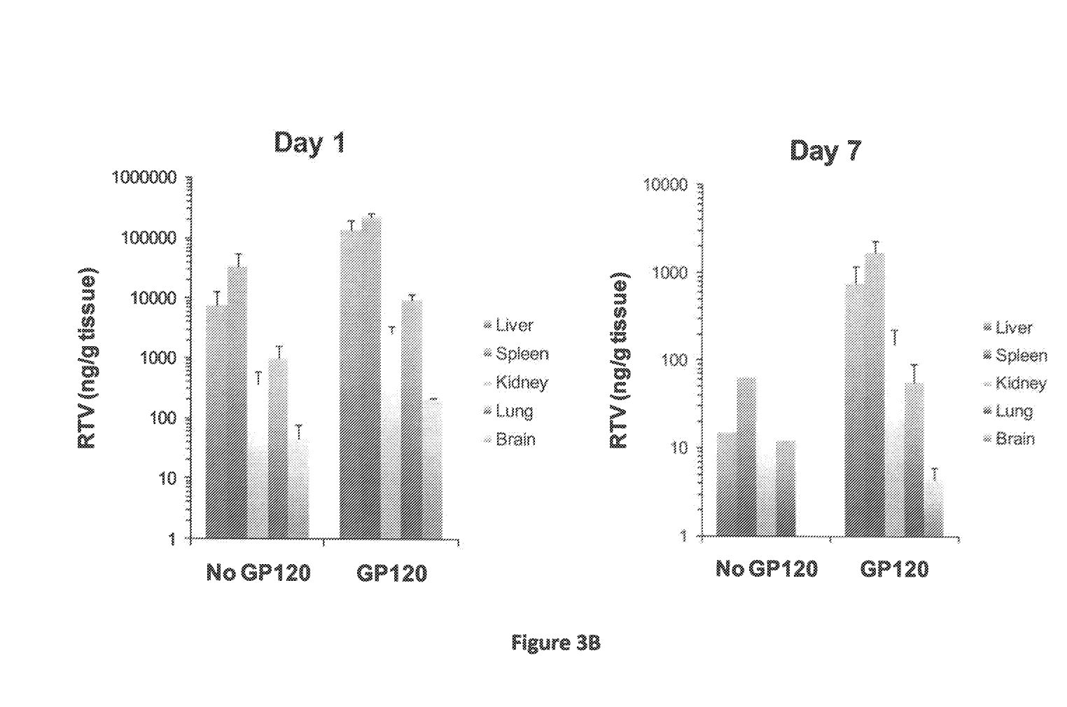

FIG. 3A shows the pharmacokinetics of gp120 nanoRTV and non-targeted nanoRTV in the plasma of mice. Gp120 nanoRTV shows increased plasma levels of RTV compared to non-targeted nanoRTV. As seen in FIG. 3A, RTV levels with gp120 nanoRTV were significantly higher (e.g., 10-100 fold) than RTV levels observed with either non-targeted nanoART particles or folic acid coated particles. Notably, this increase in vivo is significantly greater than that observed in vitro with human monocyte-derived macrophages. FIG. 3B shows the biodistribution of gp120 nanoRTV and non-targeted nanoRTV in mice. Gp120 nanoRTV shows increased tissue retention of RTV compared to non-targeted nanoRTV.

A number of publications and patent documents are cited throughout the foregoing specification in order to describe the state of the art to which this invention pertains. The entire disclosure of each of these citations is incorporated by reference herein.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A nanoparticle comprising at least one therapeutic agent and at least one surfactant linked to HIV gp120.

2. The nanoparticle of claim 1, wherein the z-average diameter is about 100 nm to 1 μm.

3. The nanoparticle of claim 1, wherein said surfactant is an amphiphilic block copolymer or phospholipid.

4. The nanoparticle of claim 1, wherein said surfactant is an amphiphilic block copolymer.

5. The nanoparticle of claim 4, wherein said amphiphilic block copolymer comprises at least one block of poly(oxyethylene).

6. The nanoparticle of claim 1, wherein said nanoparticle comprises said surfactant without linkage to HIV gp120.

7. The nanoparticle of claim 1, wherein said nanoparticle further comprises a surfactant linked to at least one other targeting ligand.

8. The nanoparticle of claim 7, wherein said targeting ligand is a macrophage or CD4+ T cell targeting ligand.

9. The nanoparticle of claim 1, wherein said therapeutic agent is an antiretroviral.

10. The nanoparticle of claim 9, wherein said antiretroviral is an anti-HIV agent.

11. The nanoparticle of claim 1, wherein said nanoparticle comprises about 5% to about 95% therapeutic agent.

12. A pharmaceutical composition comprising at least one nanoparticle of any one of claims 1 to 11 and at least one pharmaceutically acceptable carrier.

13. The pharmaceutical composition of claim 10, wherein said pharmaceutical composition further comprises at least one other anti-HIV compound.

14. A method for treating, inhibiting, and/or preventing an HIV infection in a subject in need thereof, said method comprising administering to said subject a nanoparticle of any one of claims 1 to 11.

15. The method of claim 14, further comprising the administration of at least one additional anti-HIV compound.
